(19) United States
(12) Patent Application Publication
(10) Pub. No.: US 2005/0261292 A1
(43) Pub. Date: Nov. 24, 2005

(54) Pharmaceutical composition containing N-sulfamoyl-N'-arylpiperazines for the treatment or inhibition of obesity and related conditions

(75) Inventors: Jochen Antel, Bad Muender (DE); Michael Firnges, Barsinghausen (DE); Peter-Colin Gregory, Hannover (DE); Uwe Reinecker, Ronnenberg (DE); Uwe Schoen, Burgdorf (DE); Harald Waldeck, Isernhagen (DE); Michael Wurl, Garbsen (DE); Dania Reiche, Adelheidsdorf (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 11/130,268

(22) Filed: May 17, 2005

Related U.S. Application Data
(60) Provisional application No. 60/572,236, filed on May 19, 2004.

(51) Int. Cl. A61K 31/53; A61K 31/519; A61K 31/501; A61K 31/496; A61K 31/497; C07D 43/02

(52) U.S. Cl. 514/241; 514/252.14; 514/252.16; 514/253.01; 514/252.02; 514/252.11; 514/253.05; 514/253.06; 514/254.09; 514/254.08; 544/209; 544/238; 544/279; 544/360; 544/363; 544/362

(57) ABSTRACT

The present invention relates to the use of known and novel N-sulfamoyl-N′-arylpiperazines corresponding to Formula I 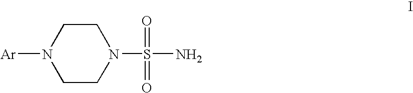 and their physiologically compatible acid addition salts for the treatment or inhibition of obesity and related conditions.

### CROSS REFERENCE TO RELATED APPLICATIONS

This application claims priority from U.S. provisional patent application No. 60/572,236, filed May 19, 2004, the entire disclosure of which is incorporated herein by reference.

### BACKGROUND OF THE INVENTION

The present invention relates to known and novel N-sulfamoyl-N′-arylpiperazines and their physiologically compatible acid addition salts and to pharmaceutical compositions or medicaments containing these compounds for the treatment or inhibition of obesity and related conditions.

Some N-sulfamoyl-N′-arylpiperazines and their use as herbicides are described in U.S. Pat. No. 3,709,677 (=DE 196 44 41). Similar compounds and their use as insecticides and acaricides are also described in international patent application no. WO 95/09151.

U.S. Pat. No. 5,728,704 (=WO 94/07867) discloses substituted 4-pyrimidine derivatives as inhibitors of sorbitol dehydrogenase, useful for treating or preventing diabetic complications in a mammal.

U.S. Pat. No. 5,215,990 (=EP 470,616) teaches substituted 4-pyrimidine derivatives useful as screening reagents for aldose reductase inhibitors.

International patent application no. WO 03/075929 provides inhibitors of histone deacetylase useful for the treatment of e.g. cancer and psoriasis, which may comprise certain N-sulfamoyl-N′-arylpiperazines. Intermediates for synthesizing said compounds are also disclosed.

U.S. Pat. No. 2,748,125 discloses 1-substituted 4-sulfamylpiperazines with anti-convulsant activity.

J. M. McManus et al. (J Med Chem 8 (1965) 766-776) teach sulfamylurea hypoglycemic agents.

A method of discovering compounds suitable for the treatment and/or inhibition of obesity by inhibiting lipogenesis via the inhibition of carbonic anhydrases in mammals and humans is known from published US patent application No. US 2002/022245 (=WO 02/07821).

### SUMMARY OF THE INVENTION

It was an object of the present invention to provide novel pharmaceutical compositions for the treatment and/or inhibition of obesity and its concomitant and/or secondary diseases or conditions, which are very effective and can be obtained in simple manner.

It has now surprisingly been found that certain novel and known N-sulfamoyl-N′-arylpiperazines or their physiologically compatible acid addition salts are suitable for the treatment and/or inhibition of obesity and its concomitant and/or secondary diseases or conditions.

According to the invention, an N-sulfamoyl-N′-arylpiperazine of general Formula I 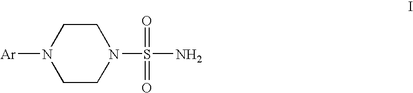
wherein

Ar is monocyclic or bicyclic C₆₋₁₀-aryl,
whose ring carbon atoms are optionally replaced one to three times by nitrogen, oxygen and/or sulfur, and/or
whose C₆₋₁₀-aryl ring system optionally contains three to five double bonds, and/or
whose C₆₋₁₀-aryl ring system is optionally substituted by one, two or three substituents which may be the same or different and which may be selected from the group consisting of halogen, carboxy, hydroxy, hydroxycarbamoyl, trifluoromethyl, cyano, nitro, pyrrolidinyl, C₁₋₄-alkyl, C₁₋₄-alkoxy, C₀₋₄-alkoxyphenyl, C₁₋₄-alkylthio, C₂₋₄-alkanoyl, C₁₋₄-alkyloxycarbonyl, C₁₋₄-alkylsulfonyl; and two oxygen atoms which are bonded to two adjacent carbon atoms of the C₆₋₁₀-aryl ring system and which are bridged by C₁₋₂-alkylene; or
whose C₆₋₁₀-aryl ring system is substituted by one or two substituents which may be the same or different and which may be selected from the group consisting of halogen, carboxy, hydroxy, hydroxycarbamoyl, trifluoromethyl, cyano, nitro, C₁₋₄-alkyl, C₁₋₄-alkoxy, C₁₋₄-alkylthio, C₂₋₄-alkanoyl, C₁₋₄-alkyloxycarbonyl, C₁₋₄-alkylsulfonyl; two oxygen atoms which are bonded to two adjacent carbon atoms of the C₆₋₁₀-aryl ring system and which are bridged by C₁₋₂-alkylene; or
whose C₆₋₁₀-aryl ring system is substituted by thienyl, naphthyl, pyridinyl; phenyl or benzyl, each of which phenyl or benzyl being optionally substituted in the phenyl ring by one, two or three substituents which may be the same or different and which may be selected from halogen, trifluoromethyl, cyano, C₁₋₆-alkyl, C₁₋₄-alkoxy or C₁₋₄-alkylsulfonyl;
   or its physiologically compatible acid addition salts can be used for the treatment and/or inhibition of obesity and its concomitant and/or secondary diseases or conditions.

More specifically, in compounds of Formula I

Ar is phenyl, optionally substituted by one, two or three substituents which may be the same or different and which may be selected from the group consisting of halogen, carboxy, hydroxy, hydroxycarbamoyl, trifluoromethyl, cyano, nitro, C₁₋₄-alkyl, C₁₋₄-alkoxy, C₀₋₄-alkoxyphenyl, C₁₋₄-alkylthio, C₂₋₄-alkanoyl, C₁₋₄-alkyloxycarbonyl, C₁₋₄-alkylsulfonyl and two oxygen atoms bonded to adjacent carbon atoms which are bridged by C₁₋₂-alkylene; or is phenyl substituted by phenyl or benzyl, each of which optionally being substituted in the phenyl ring by one or two substituents which may be the same or different and which may be selected from halogen, trifluoromethyl, C₁₋₄-alkyl and C₁₋₄-alkoxy; or
is naphthyl; pyridinyl; pyrimidinyl; pyrazinyl; pyridazinyl; triazinyl; quinolinyl; isoquinolinyl; 1,2,3,4-tetrahydroisoquinolinyl; indolyl; isoindolinyl; thieno[3,2-d]pyrimidinyl or pyrazolo[1,5-a]pyrimidinyl, each being optionally substituted by one or two substituents which may be the same or different and which may be selected from the group consisting of halogen, hydroxycarbamoyl, trifluoromethyl, cyano, nitro, pyrrolidinyl, C₁₋₄-alkyl, C₁₋₄-alkoxy and C₁₋₄-alkyloxycarbonyl.

Where in the compounds of Formula I or in other compounds described within the context of the present invention substituents are or contain C₁₋₄-alkyl or C₁₋₆-alkyl, these may each be straight-chain or branched and preferably be methyl. Where substituents in compounds of Formula I stand for halogen, fluorine, chlorine, bromine or iodine are suitable. Fluorine and chlorine are preferred. Where substituents contain C₂₋₄-alkanoyl, this may be straight-chain or branched. Acetyl is preferred as C₂₋₄-alkanoyl.

Ar preferably stands for optionally substituted phenyl; pyridinyl, in particular 2-pyridinyl or 4-pyridinyl; pyrimidinyl, in particular 2-pyrimidinyl or 5-pyrimidinyl; naphthyl or quinolinyl. Phenyl, pyridinyl and pyrimidinyl are more preferred.

Where Ar is optionally substituted phenyl, halogen, C₁₋₄-alkyl, C₁₋₄-alkoxy, trifluoromethyl, cyano, nitro and C₁₋₄-alkylsulfonyl are preferred substituents. More preferred are halogen, C₁₋₄-alkyl, C₁₋₄-alkoxy and trifluoromethyl. Unsubstituted phenyl is a preferred alternative.

Where Ar is optionally substituted pyridinyl; pyrimidinyl; naphthyl; quinolinyl; isoquinolinyl; 1,2,3,4-tetrahydroisoquinolinyl; indolyl or isoindolinyl, halogen, trifluoromethyl, cyano, C₁₋₄-alkyl and C₁₋₄-alkoxy are preferred substituents.

Particularly preferred compounds which may be used according to the invention and which are partly novel, are selected from the group consisting of 4-phenyl-piperazine-1-sulfonic acid amide (=N-sulfamoyl-N′-phenylpiperazine); 4-(2-chloro-phenyl)-piperazine-1-sulfonic acid amide; 4-(2-methoxy-phenyl)-piperazine-1-sulfonic acid amide; 4-pyridin-4-yl-piperazine-1-sulfonic acid amide; 4-pyrimidin-2-yl-piperazine-1-sulfonic acid amide; 4-(4-fluoro-phenyl)-piperazine-1-sulfonic acid amide; 4-(4-chloro-3-trifluoromethyl-phenyl)-piperazine-1-sulfonic acid amide and 4-(3-chloro-5-trifluoromethyl-pyridin-2-yl)-piperazine-1-sulfonic acid amide.

Physiologically compatible acid addition salts of compounds of Formula I are their conventional salts with inorganic acids, for example sulfuric acid, phosphoric acids or hydrohalic acids, preferably hydrochloric acid, or with organic acids, for example lower aliphatic monocarboxylic, dicarboxylic or tricarboxylic acids such as maleic acid, fumaric acid, lactic acid, tartaric acid, citric acid, or with sulfonic acids, for example lower alkanesulfonic acids such as methanesulfonic acid or trifluoromethanesulfonic acid, or benzenesulfonic acids optionally substituted in the benzene ring by halogen or lower alkyl, such as p-toluenesulfonic acid. Hydrochloric acid salts of the compounds of Formula I are preferred.

In a further aspect, the invention also relates to compounds of general Formula Ia, 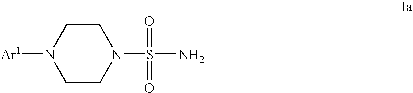
wherein

Ar¹ is phenyl, optionally substituted by one, two or three substituents which may be the same or different and which may be selected from the group consisting of halogen, carboxy, hydroxy, hydroxycarbamoyl, trifluoromethyl, cyano, nitro, C₁₋₄-alkyl, C₁₋₄-alkoxy, C₀₋₄-alkoxyphenyl, C₁₋₄-alkylthio, C₂₋₄-alkanoyl, C₁₋₄-alkyloxycarbonyl, C₁₋₄-alkylsulfonyl and two oxygen atoms bonded to adjacent carbon atoms which are bridged by C₁₋₂-alkylene; or is phenyl substituted by phenyl or benzyl, each of which optionally being substituted in the phenyl ring by one or two substituents which may be the same or different and which may be selected from halogen, C₁₋₄-alkyl, C₁₋₄-alkoxy and trifluoromethyl; or
is naphthyl; pyridinyl; 2-pyrimidinyl; 5-pyrimidinyl; pyrazinyl; pyridazinyl; triazinyl; quinolinyl; isoquinolinyl; 1,2,3,4-tetrahydroisoquinolinyl; indolyl; isoindolinyl; thieno[3,2-d]pyrimidinyl or pyrazolo[1,5-a]pyrimidinyl each optionally being substituted by one or two substituents which may be the same or different and which may be selected from the group consisting of halogen, hydroxycarbamoyl, trifluoromethyl, cyano, nitro, pyrrolidinyl, C₁₋₄-alkyl, C₁₋₄-alkoxy and C₁₋₄-alkyloxycarbonyl;
   and their physiologically compatible acid addition salts, for the use as medicament for mammals and humans; and/or to pharmaceutical compositions comprising a pharmacologically effective quantity of a compound of Formula Ia or its physiologically compatible acid addition salts and conventional pharmaceutically acceptable auxiliaries and/or carriers.

In still a further aspect, the present invention relates to novel N-sulfamoyl-N′-arylpiperazines of general Formula Ib, 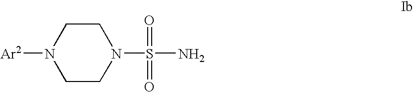
wherein

Ar² is phenyl substituted once by fluoro, 3-chloro, 4-chloro, bromo, iodo, hydroxy, C₁₋₄-alkyl, C₂₋₄-alkoxy, C₀₋₄-alkoxyphenyl, C₁₋₄-alkylthio, C₂₋₄-alkanoyl, C₁₋₄-oxycarbonyl, hydroxycarbamoyl, carboxy, trifluoromethyl, cyano, nitro, two oxygen atoms bonded to adjacent carbon atoms which are bridged by C₁₋₂-alkylene, and C₁₋₄-alkylsulfonyl; or
is phenyl substituted by two or three substituents which may be the same or different and which may be selected from the group consisting of halogen, carboxy, hydroxy, hydroxycarbamoyl, trifluoromethyl, cyano, nitro, C₁₋₄-alkyl, C₁₋₄-alkoxy, C₁₋₄-alkylthio, C₂₋₄-alkanoyl, C₁₋₄-oxycarbonyl, C₁₋₄-alkylsulfonyl and two oxygen atoms bonded to adjacent carbon atoms which are bridged by C₁₋₂-alkylene; or
is phenyl substituted once by phenyl or benzyl, each of which optionally being substituted in the phenyl ring by one or two substituents which may be the same or different and which may be selected from halogen, trifluoromethyl, C₁₋₄-alkyl and C₁₋₄-alkoxy; or
is naphthyl; pyridinyl; 2-pyrimidinyl; 5-pyrimidinyl; pyrazinyl; pyridazinyl; triazinyl; quinolinyl; isoquinolinyl; indolyl; isoindolinyl; thieno[3,2-d]pyrimidinyl or pyrazolo[1,5-a]pyrimidinyl each optionally being substituted by one or two substituents which may be the same or different and which may be selected from the group consisting of halogen, hydroxycarbamoyl, trifluoromethyl, cyano, nitro, pyrrolidinyl, C₁₋₄-alkyl, C₁₋₄-alkoxy and C₁₋₄-oxycarbonyl; or
is 1,2,3,4-tetrahydroisoquinolinyl substituted by one or two substituents which may be the same or different and which may be selected from the group consisting of halogen, hydroxycarbamoyl, trifluoromethyl, cyano, nitro, pyrrolidinyl, C₁₋₄-alkyl, C₁₋₄-alkoxy and C₁₋₄-oxycarbonyl;
   and their physiologically compatible acid addition salts.

Some N-sulfamoyl-N′-arylpiperazines which are coming under the scope of general Formula I according to the present invention are already known, e.g. from patent applications DE-OS 1964441 (U.S. Pat. No. 3,709,677), WO 94/07867 and/or WO 95/09151, and can be produced according to the processes described in these specifications or according to analogous processes.

In general, compounds of Formula I (including compounds of Formulas Ia and Ib) can be produced in known manner by either

a) reacting an arylpiperazine compound of general Formula II, 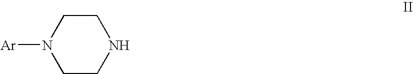
wherein Ar has the above meaning, with sulfamide, or

b) reacting an arylpiperazine of Formula II with a 4-dimethylaminopyridin (=DMAP) reagent which is protected with the tert.-butyloxycarbonyl (=boc) group, of Formula III, 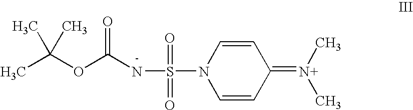
and subsequently cleaving off the boc group under acidic conditions from the obtained intermediate compound, or

c) reacting an arylpiperazine of Formula II with sulfamoylchloride, which is preferably protected with the boc group, of Formula IV, 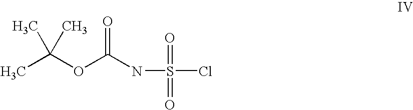
and subsequently cleaving off the boc group under acidic conditions from the obtained intermediate product,
and if desired converting resulting free bases of Formula I into their physiologically compatible salts, or converting salts of the compounds of Formula I into the free bases of Formula I.

In process variant a), the reaction can be carried in an organic solvent which is inert under the reaction conditions, in particular in an aprotic solvent such as toluene or xylene or in a mixture of such solvents. Suitable reaction temperatures are between room temperature and the boiling point of the solvent or solvent mixture, preferably between 60° C. and 100° C.

In process variant b), the reaction can be carried out in an organic solvent which is inert under the reaction conditions, in particular a dipolar-aprotic solvent such as chloroform, dichloromethane or dioxane, or in a mixture of such solvents. Suitable reaction temperatures are between 10° C. and 50° C., preferably at room temperature. The boc protecting group can subsequently be cleaved off in a known manner in acidic media, e.g. in an ethanolic solution of hydrochloric acid.

In process variant c), the reaction can be carried out in an organic solvent which is inert under the reaction conditions, in particular a dipolar-aprotic solvent such as chloroform or dichloromethane or in a mixture of such solvents. Suitable reaction temperatures are between 10° C. and 50° C., preferably at room temperature. The boc protecting group can subsequently be cleaved off in a known manner in acidic media, e.g. in an ethanolic solution of hydrochloric acid. Using a boc-protected sulfamoylchloride is preferred. However, under the appropriate reaction conditions known to those skilled in the art, also unprotected sulfamoylchloride in the presence of a base may be used.

Compounds of Formula II are generally known compounds or such compounds can routinely be prepared by those skilled in the art according to known processes and from known starting materials. For example can compounds of Formula II, wherein Ar is optionally substituted biaryl, be prepared by reacting a compound of general Formula V, 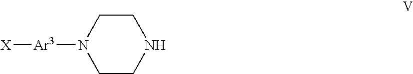
wherein Ar³ has the meaning monocyclic or bicyclic C₆₋₁₀-aryl, whose ring carbon atoms are optionally replaced one to three times by nitrogen, oxygen and/or sulfur, and/or whose C₆₋₁₀-aryl ring system optionally contains three to five double bonds; and X is a cleavable leaving group like halogen, preferably bromine; with a compound of general Formula VI, 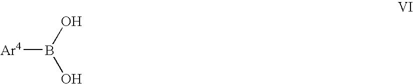
wherein Ar⁴ has the meaning thienyl, naphthyl, pyridinyl; phenyl or benzyl, each of which phenyl or benzyl being optionally substituted in the phenyl ring by one, two or three substituents which may be the same or different and which may be selected from halogen, trifluoromethyl, cyano, C₁₋₆-alkyl, C₁₋₄-alkoxy or C₁₋₄-alkylsulfonyl; in the presence of a palladium catalyst.

The reaction can be carried out in a manner known as “Suzuki coupling reaction” in an organic solvent which is inert under the reaction conditions, in particular in a dipolar-protic solvent such as a lower alkanol like methanol or ethanol, or an ether of a lower divalent alkanol like ethylene glycol dimethyl ether, or in mixtures of such solvents or in mixtures of such solvents with water. Suitable reaction temperatures are between 100° C. and 200° C., preferably between 120° C. and 180° C. The reaction can advantageously be carried out by using a microwave reactor. Usually, the reaction is carried out in the presence of a base like an alkalicarbonate, preferably potassium carbonate. Suitable palladium catalysts are salts of palladium-(II), like palladium-(II)-acetate.

Compounds of Formulas III and IV are generally known compounds and can routinely be prepared by those skilled in the art according to known processes and from known starting materials. Compounds of Formula V and VI are generally known compounds or such compounds can routinely be prepared by those skilled in the art according to known processes and from known starting materials.

In yet another aspect, the present invention also relates to a method of treating or inhibiting obesity, the metabolic syndrome and/or syndrome X and/or cardiovascular diseases, in mammals and humans comprising administering to a subject in need thereof a therapeutically effective amount of a compound of Formula I or its physiologically compatible acid addition salts.

Obesity according to the present invention is meant to comprise any increase in body fat that results in increased bodyweight, comprising as a preferred alternative but not limited to the medical definition of obesity. The invention thus also relates to non-medical weight loss, such as cosmetic weight loss and includes improving bodily appearance in general. Further, the term obesity also is meant to comprise drug induced obesity and/or juvenile obesity.

The concomitant diseases of obesity or the secondary diseases thereof which can each be treated with the compounds according to the invention include in particular the metabolic syndrome and/or syndrome X and cardiovascular diseases.

The term “metabolic syndrome” as used in this application is meant to cover a complex of clinical pictures which—besides central obesity—mainly comprises hypertension, in particular arterial hypertension; insulin resistance, in particular diabetes mellitus type II; glucose intolerance; dyslipoproteinaemia, in particular as hypertriglyceridaemia, accompanied by dyslipoproteinaemia occurring with lowered HDL-cholesterol, and also hyperuricaemia, which can lead to gout. According to information from the American Heart Association, the metabolic syndrome is closely linked to insulin resistance. Some people are genetically predisposed to insulin resistance. Acquired factors, such as excess body fat and physical inactivity, can elicit insulin resistance and the metabolic syndrome in these people. Most people with insulin resistance have central obesity. The biologic mechanisms at the molecular level between insulin resistance and metabolic risk factors are not fully understood and appear to be complex. One group of people at risk for developing metabolic syndrome are those with diabetes who have a defect in insulin action and cannot maintain a proper level of glucose in their blood. Another is people, mainly those with high blood pressure, who are nondiabetic and insulin-resistant but who compensate by secreting large amounts of insulin. This condition is known as hyperinsulinemia. A third group is heart attack survivors who, unlike hypertensives, have hyperinsulinemia without having abnormal glucose levels. The metabolic syndrome has become increasingly common in higher developed countries like the United States, where it is estimated that about 20-25 percent of US adults have it. There are no well-accepted criteria for diagnosing the metabolic syndrome.

The criteria proposed by the Third Report of the National Cholesterol Education Program (NCEP) Expert Panel on Detection, Evaluation, and Treatment of High Blood Cholesterol in Adults (Adult Treatment Panel III) are the most current and widely used. According to the ATP III criteria, the metabolic syndrome is identified by the presence of three or more of these components:
Central obesity as measured by waist circumference (Men—Greater than 40 inches; Women—Greater than 35 inches).
Fasting blood triglycerides greater than or equal to 150 mg/dL.
Blood HDL cholesterol (Men—Less than 40 mg/dL; Women—Less than 50 mg/dL)
Blood pressure greater than or equal to 130/85 mmHg.
Fasting glucose greater than or equal to 110 mg/dL.

The term “syndrome X” is closely related to the term “metabolic syndrome” and usually is supposed to denominate the identical disease or condition. According to information from the American Heart Association, the term “Syndrome X” refers, however, additionally to a heart condition where chest pain and electrocardiographic changes that suggest ischemic heart disease are present, but where there are no angiographic findings of coronary disease. Patients with cardiac syndrome X also sometimes have lipid abnormalities.

The term “cardiovascular diseases” in conjunction with obesity is usually understood to mean coronary heart disease, which can lead to heart failure, cerebrovascular diseases, which may for example be accompanied by an increased risk of strokes, and peripheral occlusive arterial disease.

Due to their inherent properties, the compounds of Formula I or their physiologically compatible acid addition salts are also expected to be useful in the treatment of diabetic conditions or diseases which are unrelated to obesity. Such diabetic conditions or diseases comprise e.g. diabetes mellitus type II, diabetic neuropathy, diabetic retinopathy, diabetic nephropathy, diabetic microangiopathy or diabetic macroangiopathy.

Further concomitant and/or secondary diseases of obesity may be gall-bladder diseases such as formation of gallstones, sleep apnoea syndrome, orthopaedic complications such as osteoarthritis and psychosocial disorders.

The compounds of Formula I are further deemed to be useful as anticonvulsants for the treatment or inhibition of epilepsy in mammals and humans.

The compounds of Formula I according to the invention are inhibitors of mammalian carbonic anhydrases, in particular of human carbonic anhydrase isozymes of subtypes II and/or V (=hCA II and/or hCA V).

Pharmacological Test Methods

The example numbers quoted in the pharmacological test methods relate to the preparation examples described below.

1. In vitro Inhibition of Human Carbonic Anhydrase Isoenzyme II (hCA II)

The test compounds of Formula I in 96 well microplates were diluted with aqua bidest by using an automatic pipettor (CyBiWell®). From the different dilution plates, aliquots of 20 μl were transferred to the 96 well black assay plates with a pipetting station (Tecan Genesis®). In a second step, 148 μl of potassium phosphate buffer (20 mM, pH 7.4) was added, and as a third step, 20 μl of enzyme solution (1 μM human carbonic anhydrase isoenzyme II from erythrocytes (Sigma-Aldrich), dissolved in potassium phosphate buffer) incubated for 60 min at room temperature and the fluorescence signal (Tecan Ultra® fluorescence reader; excitation wavelength: 280 nm; emission wavelength: 465 nm) read at the end of the preincubation period (FLU-1). After the preincubation time, 20 μl of aqueous dansylamide solution (1 mM dansylamide (Sigma-Aldrich), dissolved in hydrochloric acid) were added and the fluorescence signal read every 10 min for a period of 60 min at 37° C. For calculation, the fluorescence data of the time point 60 min (FLU-2) were used. The total volume of assay mixture amounted to 208 μl. The final concentration of carbonic anhydrase II was 10⁻⁷ M/L, of dansylamide 2.25×10⁻⁶ and of compounds from 10⁻⁸ M/L up to 10⁻⁵ M/L. Final concentration of DMSO as compound solvent was 0.1 mM. Each microplate also contained blanks without compound and enzyme, controls without compound and ethoxzolamide (final concentration 5×10⁻⁸ M/L). All data reflect single measurements. Data were expressed as % inhibition after calculation by the formula:
% inhibition=100((1−(FLU−2_{cpd}−FLU−2_{blank}−FLU−1_{cpd}+FLU−1_{blank})/(FLU−2_{control}−FLU−2_{blank}−FLU−1_{control}−FLU−1_{blank}))

The % inhibition data for each compound and the respective final concentrations were used for IC₅₀ calculations by using the Prism 4 software. Concentration action figures were calculated by applying the Prism algorithm for nonlinear regression (curve-fit): sigmoidal dose response with variable slope and the constraints: top: 100 and bottom 0.

In this test model, the test substances of Formula I listed in Table 1 below showed the IC₅₀ values given below:

TABLE 1
hCA II inhibiting effect of the test substances in vitro

| Example No. | IC₅₀ [μM] |
|---|---|
| 2 | 5.1 |
| 3 | 7.8 |
| 4 | 3.7 |
| 7 | 1.8 |
| 8 | 1.7 |
| 9 | 7.3 |
| 10 | 4.0 |
| 11 | 0.5 |
| 12 | 1.2 |
| 13 | 0.9 |
| 14 | 0.2 |
| 15 | 0.4 |
| 16 | 0.3 |
| 17 | 1.9 |
| 18 | 1.6 |
| 19 | 0.08 |
| 20 | 0.3 |
| 21 | 0.2 |

2. In vivo Oral Glucose Tolerance Test in the Rat

The studies were carried out in individually housed male fatty Zucker rats (n=10 per group) weighing ca. 250-500 g. The rats were kept on a normal 12/12 h light/dark cycle (lights on 07.00) and they were allowed food (lab chow) and water ad libitum except for during experiments, when they were fasted overnight before the glucose challenge.

The test substances of Formula I were suspended in 2% polyethylenglycol (=PEG) 1% carboxymethylcellulose and administered by oral gavage at a dose of 100 mg/kg/day; ⅓ of the dose (1 ml/kg, 33 mg/ml) was administered at 08.30-09.30 h; the remaining ⅔ dose (2 ml/kg; 33 mg/ml) was administered between 16.00-17.00 h and the last ⅓ dose was given the following morning. Control animals received only the vehicle. On the day of the test, 45 min after the final dose of test substance/vehicle a blood sample (0 min) was taken (tail vein) immediately after which the rats received an oral glucose challenge (1.25 g/kg; 118 mg/ml). Further blood samples were taken at 30, 60, 90, 120 min after the glucose challenge. The second drop of blood of each sample was placed on a glucose test strip before this was placed in the glucose meter for determination of blood glucose level (Life Scan One Touch Ultra Blood® Glucose Meter and Life Scan One Touch Ultra® Test Strips; Life Scan Inc.; Milpitas, Calif. 95035). The remaining blood of each sample was spun and the plasma was frozen at −80° C. before analysis for insulin (1-2-3 Rat Insulin ELISA kit, Alpco Diagnostics).

The values obtained were plotted and the AUC for test compounds and vehicle (for glucose and insulin) were determined after which the percent control AUC, percent control maximum value and % control baseline were estimated, to determine the influence of the test compound on the glucose tolerance.

In the test model described above, the test substances showed the following results (given as percentage % of control):

TABLE 2
Influence of test substances on glucose and insulin levels

| | Glucose | | | Insulin | | |
|---|---|---|---|---|---|---|
| Example | | Maximal | | | Maximal | |
| No. | AUC | effect | Baseline | AUC | effect | Baseline |
| 2 | 84 | 78 | 82 | 95 | 95 | 85 |
| 6 | 89 | 84 | 91 | 104 | 123 | 109 |
| 14 | 102 | 94 | 88 | 107 | 92 | 97 |

3. Acute in vivo Food Intake Test in Mice

The studies were carried out in individually housed male C57Bl/6 mice (n=8 per group). The mice were kept on an inverted 12/12 h light/dark cycle (lights on 22:00). They were allowed food (high caloric diet) and water ad libitum. Food intake and water consumption was measured daily. The test compound of Formula I was suspended in 1% methylcellulose in water and 2% (v/v) of Poloxamer 188 (Lutrol F68®) and administered by oral gavage at a dose of 100 mg/kg/day. One half of the dose was administered at 7.00-9.00 h; the remaining half of the dose was administered between 15.00-15.30 h.

In the test model described above, the test substances caused a decrease of the animals' 24 h food intake to the percentages of food intake when compared to control as given in table 3 below.

TABLE 3
Influence of test substances on food intake

| | food intake |
|---|---|
| Example No. | [% of control] |
| 2 | 68 |
| 7 | 78 |
| 12 | 53 |
| 15 | 79 |
| 21 | 76 |

4. Chronic Influence on Food and Water Intake and Body Weight Gain in vivo

Female Wistar rats (weight range 250-300 g; Charles River, Margate, Kent) were housed in pairs in polypropylene cages with solid floors and sawdust bedding at a temperature of 21±4° C. and 55±20% humidity. Animals were maintained on a reverse phase light-dark cycle (lights off for 8 hours from 10.00-18.00 h) during which time the room was illuminated by red light. Animals had free access to powdered high fat diet (VRF1 plus 20% lard), ground chocolate, ground peanuts and tap water at all times. The three different diets were contained in separate glass feeding jars with aluminium lids (Solmedia Laboratory Suppliers, Romford, Essex). Each lid had a 3-4 cm hole cut in it to allow access to the food. Animals were housed in pairs for twelve weeks. At least two weeks before the start of the baseline readings, animals were housed individually in polypropylene cages with wire grid floors to enable the food intake of each rat to be recorded. Polypropylene trays with cage pads were placed beneath each cage to detect any food spillage.

At the start of the study, animals were weighed (to the nearest 0.1 g using an electronic top-pan balance) and allocated into 6 weight-matched treatment groups, each containing 10 animals. Following a 7 day baseline run-in period, during which time all animals were dosed orally once a day with vehicle (1% Tylose MH50, 0.1% poloxamer 188), rats were given vehicle or test compound of Formula I for 28 days as described in Table 4 below:

TABLE 4
Treatment scheme of rats with compound of
Formula I, example 2 (= ex. 2)

| Group | Treatment 1 (0 h) | Treatment 2 (4 h) | n |
|---|---|---|---|
| A | Vehicle po | Vehicle po | 10 |
| B | Ex. 2, 30 mg/kg po | Ex. 2, 30 mg/kg po | 10 |
| C | Ex. 2, 50 mg/kg Po | Ex. 2, 50 mg/kg po | 10 |

The test substance of example 2 was suspended in 1% Tylose MH50, 0.1% poloxamer 188 and administered by oral gavage (2 ml/kg). All dosing occurred at the onset of the 8 hours dark period (spanning the period immediately before and after lights out). The second treatments were given 4 hours after the first. Rats, feeding jars and water bottles were weighed (to the nearest 0.1 g) every day at the time of administration of vehicle or test substance. At each reading, the tray below each cage was examined for spilt food, which was returned to the appropriate jar before weighing. However, spillage of food from the feeding jars was negligible. Variations in body weight and energy levels of the different types of food were accounted for by expressing the food intake results in terms of kJ/kg rat weight. Water intake results were expressed in g/kg.

Animals were killed at the end of the study (by CO₂ to minimize any fluid loss) on day 29 and blood samples (5 ml whole blood/animal) were collected by cardiac puncture. Plasma was separated by centrifugation and stored at −75° C. until analysis. Following blood collection carcasses were weighed, frozen and stored at −75° C. for body composition analysis. Body fat, protein, water and ash levels of the carcasses were determined using standard chemical analysis techniques. Only fat, protein, water and ash content were measured as other components (mainly carbohydrate) form less than 2% of total body composition.

Carcasses were individually milled at the temperature of liquid nitrogen, mixed and two representative samples taken. Carcass water was determined by freeze-drying the samples to constant weight. Carcass fat was determined on the freeze-dried samples using a modified Soxhlet extraction protocol (petroleum ether at 40-60° C.) with a Foss Soxtec® HT2 system (Foss UK Ltd, Wheldrake, UK) according to the manufacturers recommended protocol. Carcass protein was determined using a micro-Kjeldahl procedure on the freeze-dried samples using a Foss 2012® digestion block and Foss 2200® distilling unit (Foss UK Ltd). Residual carcass ash was determined by firing the freeze-dried samples at high temperatures using a muffle ashing furnace. Repeat determinations of the chemical analysis parameters were performed as necessary (e.g. if the duplicate samples differed by more than 1%).

TABLE 5
Effect of the test compound of example 2 on water, fat, protein and
ash content of carcasses: mean weight per rat

| Group | Water (g) | Fat (g) | Protein (g) | Ash (g) | Carcass (g) |
|---|---|---|---|---|---|
| Vehicle | 210.9 ± 2.7 | 144.5 ± 11.6 | 67.5 ± 1.2 | 12.52 ± 0.45 | 438.4 ± 12.1 |
| Example 2 | 205.8 ± 3.1 | 87.9 ± 3.8** | 64.8 ± 1.2 | 12.13 ± 0.48 | 375.4 ± 4.8** |
| (30 mg/kg) | | | | | |
| Example 2 | 202.2 ± 3.6 | 83.6 ± 6.9** | 62.8 ± 1.3 | 11.38 ± 0.29* | 365.6 ± 3.8** |
| (50 mg/kg) | | | | | |

Results are expressed as treatment group means (all n = 9-10 and adjusted for differences between the groups in body weight at baseline) and standard error of the mean (=SEM; calculated from the residuals of the statistical model). Statistical comparisons were by ANCOVA (baseline body weight as covariate) followed by Williams' test.
Significant differences are denoted by
**p < 0.001,
*° p < 0.01,
*p < 0.05 vs vehicle.

TABLE 6
Effect of the test compound of example 2 on water, fat, protein and ash
content of carcasses: mean percent final body weight

| Group | Water (%) | Fat (%) | Protein (%) | Ash (%) | Total (%) |
|---|---|---|---|---|---|
| Vehicle | 48.8 ± 1.0 | 31.9 ± 1.4 | 15.7 ± 0.4 | 2.91 ± 0.12 | 99.3 |
| Example 2 | 55.2 ± 0.8** | 22.8 ± 1.0** | 17.4 ± 0.3*° | 3.24 ± 0.12 | 98.6 |
| (30 mg/kg) | | | | | |
| Example 2 | 55.7 ± 1.2** | 22.3 ± 1.8** | 17.3 ± 0.4*° | 3.14 ± 0.1 | 98.4 |
| (50 mg/kg) | | | | | |

Results and statistics are expressed as above.

TABLE 7
Effect of test compound of Example 2 on plasma
parameters in dietary obese female wistar rats

| Group | Glucose (mM) | Insulin (ng/ml) | Leptin (ng/ml) |
|---|---|---|---|
| Vehicle | 8.2 ± 0.7 | 2.33 ± 0.53 | 144.6 ± 22.7 |
| Ex. 2 (30 mg/kg) | 8.6 ± 0.5 | 1.53 ± 0.33 | 68.8 ± 9.4** |
| Ex. 2 (50 mg/kg) | 7.9 ± 0.5 | 1.40 ± 0.41 | 58.8 ± 11.6** |

Results and statistics are expressed as above.

The present invention further provides a pharmaceutical composition or medicament comprising a pharmacologically effective quantity of a compound of Formula I or its physiologically compatible acid addition salts and preferably further comprising conventional pharmaceutically acceptable auxiliaries and/or carriers.

Suitable pharmaceutically acceptable auxiliaries and/or carriers are well known in the art and include pharmaceutical grade starch, mannitol, lactose, magnesium stearate, sodium saccharin, talcum, cellulose, glucose, sucrose (or other sugar), magnesium carbonate, gelatin, oil, alcohol, detergents, emulsifiers or water (preferably sterile). The composition may be a mixed preparation of a composition or may be a combined preparation for simultaneous, separate or sequential use (including administration). The compounds according to the invention or their physiologically compatible acid addition salts for use in the aforementioned indications may be administered by any convenient method, for example by oral (including by inhalation), parenteral, mucosal (e.g. buccal, sublingual, nasal), rectal or transdermal administration and the compositions adapted accordingly. For oral administration, the compounds can be formulated as liquids or solids, for example solutions, syrups, suspensions or emulsions, tablets, capsules and lozenges. A liquid formulation will generally consist of a suspension or solution of the compound or physiologically acceptable salt in a suitable aqueous or non-aqueous liquid carrier(s) for example water, ethanol, glycerine, polyethylene glycol or an oil.

The formulation may also contain a suspending agent, preservative, flavoring or coloring agent. A composition in the form of a tablet can be prepared using any suitable pharmaceutical carrier(s) routinely used for preparing solid formulations. Examples of such carriers include magnesium stearate, starch, lactose, sucrose and microcrystalline cellulose. A composition in the form of a capsule can be prepared using routine encapsulation procedures. For example, powders, granules or pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule. Compositions for oral administration may be designed to protect the active ingredient against degradation as it passes through the alimentary tract, for example by an outer coating of the formulation on a tablet or capsule. Typical parenteral compositions consist of a solution or suspension of the compound or physiologically compatible acid addition salts in a sterile aqueous or non-aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration. Compositions for nasal or oral administration may conveniently be formulated as aerosols, drops, gels and powders.

Aerosol formulations typically comprise a solution or fine suspension of the active substance in a physiologically acceptable aqueous or non-aqueous solvent and are usually presented in single or multi-dose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a pharmaceutically acceptable propellant. The aerosol dosage forms can also take the form of a pump-atomizer. Compositions suitable for buccal or sublingual administration include tablets, lozenges and pastilles, wherein the active ingredient is formulated with a carrier such as sugar and acacia, tragacanth, or gelatin and glycerin. Compositions for rectal or vaginal administration are conveniently in the form of suppositories (containing a conventional suppository base such as cocoa butter), pessaries, vaginal tabs, foams or enemas. Compositions suitable for transdermal administration include ointments, gels, patches and injections including powder injections. Conveniently the composition is in unit dose form such as a tablet, capsule or ampoule. The pharmaceutical compositions according to the invention are useful in the prevention and/or treatment of obesity, concomitant and/or secondary diseases of obesity; other medical weight loss and non-medical related weight loss; and/or diabetic conditions or diseases.

The compounds of the present invention and their physiologically compatible acid addition salts are generally administered as pharmaceutical compositions which are important and novel embodiments of the invention because of the presence of the compounds disclosed herein. In embodiments of the invention, a pharmaceutical pack or kit is provided comprising one or more container(s) filled with one or more of the ingredients of a pharmaceutical composition of the invention. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration.

Yet a further aspect of the invention provides a process for the manufacture of a pharmaceutical composition as described hereabove. The manufacture can be carried out by standard techniques well known in the art and involves combining a compound according to the invention and the pharmaceutically acceptable auxiliaries and/or carriers. The composition may be in any form including a tablet, a liquid, a capsule, and a powder or in the form of a food product, e.g. a functional food. In the latter case the food product itself may act as the pharmaceutically acceptable carrier.

The compound or composition is preferably administered to a patient in need thereof and in a quantity sufficient to prevent and/or treat the symptoms of the condition, disorder or disease For all aspects of the invention, particularly medical ones, the administration of a compound or composition has a dosage regime which will ultimately be determined by the attending physician and will take into consideration such factors such as the compound being used, animal type, age, weight, severity of symptoms, method of administration, adverse reactions and/or other contraindications. Specific defined dosage ranges can be determined by standard design clinical trials with patient progress and recovery being fully monitored. Such trials may use an escalating dose design using a low percentage of the maximum tolerated dose in animals as the starting dose in man. The physiologically acceptable compounds of the invention will normally be administered in a daily dosage regimen (for an adult patient) of, for example, an oral dose of between 1 mg and 2000 mg, preferably between 30 mg and 1000 mg, e.g. between 10 and 250 mg or an intravenous, subcutaneous, or intramuscular dose of between 0.1 mg and 100 mg, preferably between 0.1 mg and 50 mg, e.g. between 1 and 25 mg of the compound of the Formula I or a physiologically acceptable salt thereof calculated as the free base, the compound being administered 1 to 4 times per day. The compound used according to the invention can also be administered to children or juveniles while the individual dosage regimens in these cases will need to be particularly thoroughly adjusted by the physician and will usually comprise lower doses than will be administered to adults.

Suitably the compounds will be administered for a period of continuous therapy, for example for at least a week, but usually for a longer period of several weeks to several months. The invention also provides a cosmetic method (non-therapeutic) for maintaining a given weight, or for cosmetic weight loss, the method comprising the administration of a compound according to the other aspects of the invention, preferably in combination with a pharmaceutically acceptable carrier or diluent.

The compound or composition is preferably administered to a subject in need or in desideratum thereof and in a quantity sufficient to maintain a given weight or for cosmetic weight loss.

In still a further aspect, the compounds of Formula I and their physiologically compatible acid addition salts may favourably be administered in combination with one or more active agents (as a pharmaceutical combination composition) selected from antidiabetics; antiobesity or appetite-regulating agents; cardiovascular active agents, in particular antihypertensives; diuretics; active agents altering lipid levels, in particular lipid-lowering agents; and active ingredients for the treatment and/or prevention of complications caused by diabetes or associated with diabetes.

Suitable antidiabetics comprise e.g. insulins, amylin, derivatives of GLP-1 and GLP-2 such as, for example, those disclosed in WO 98/08871 and orally active hypoglycemic active ingredients. The orally active hypoglycemic active ingredients preferably comprise sulfonylureas, e.g tolbutamide, glibenclamide, glimepiride, glipizide, gliquidone, glisoxepide, glibomuride or gliclazide; biguanides, e.g. metformin; meglitinides, e.g. repaglinide; beta3 adrenergic agonists; oxadiazolidinediones; glucosidase inhibitors e.g. alpha-glucosidase inhibitors such as miglitol or acarbose; glucagon receptor antagonists, GLP-1 agonists, potassium channel openers like diazoxide or those disclosed in WO 97/26265 or WO 99/03861; CB-1 (cannabinoid-1 receptor) antagonists/inverse agonists; insulin sensitizers like thiazolidinediones, e.g. troglitazone, ciglitazone, pioglitazone, rosiglitazone or the compounds disclosed in WO 97/41097, in particular 5-[[4-[(3,4-dihydro-3-methyl-4-oxo-2-quinazolinylmethoxy]phenyl]methyl]-2,4-thiazolidinedione; activators of insulin receptor kinase; inhibitors of liver enzymes involved in the stimulation of gluconeogenesis and/or glycogenolysis, for example inhibitors of glycogen phosphorylase; and modulators of glucose uptake and glucose excretion.

Suitable antiobesity or appetite-regulating agents comprise one or more of a 5-HT (serotonin) transporter inhibitor, a NE (norepinephrine) transporter inhibitor, a CB-1 (cannabinoid-1 receptor) antagonist/inverse agonist, a ghrelin antibody, a ghrelin antagonist, a H3 (histamine H3) antagonist/inverse agonist, a MCH1R (melanin concentrating hormone 1R) antagonist, a MCH2R (melanin concentrating hormone 2R) agonist/antagonist, a NPY1 (neuropeptide Y Y1) antagonist, a NPY2 (neuropeptide Y Y2) agonist, a NPY5 (neuropeptide Y Y5) antagonist, leptin, a leptin derivative, an opioid antagonist, an orexin antagonist, a BRS3 (bombesin receptor subtype 3) agonist, a CCK-A (cholecystokinin-A) agonist, a CNTF (ciliary neurotrophic factor), a CNTF derivative, a GHS (growth hormone secretagogue receptor) agonist, SHT2c (serotonin receptor 2c) agonist, a Mc3r (melanocortin 3 receptor) agonist, a Mc4r (melanocortin 4 receptor) agonist, a monoamine reuptake inhibitor, a serotonin reuptake inhibitor, a GLP-1 (glucagon-like peptide 1) agonist, topiramate, phytopharm compound 57, an ACC2 (acetyl-CoA carboxylase-2) inhibitor, a beta3 adrenergic agonist, a DGAT1 (diacylglycerol acyltransferase 1) inhibitor, a DGAT2 (diacylglycerol acyltransferase 2) inhibitor, a FAS (fatty acid synthase) inhibitor, a PDE (phosphodiesterase) inhibitor, a thyroid hormone B agonist, an UCP-1 (uncoupling protein 1), 2, or 3 activator, an acyl-estrogen, a glucocorticoid antagonist, an 11 HSD-1 (11-beta hydroxy steroid dehydrogenase type 1) inhibitor, a SCD-1 (stearoyl-CoA desaturase-1) inhibitor, a dipeptidyl peptidase IV (DP-IV) inhibitor, a lipase inhibitor, a fatty acid transporter inhibitor, a dicarboxylate transporter inhibitor, a glucose transporter inhibitor, a phosphate transporter inhibitor, and pharmaceutically acceptable salts and esters thereof.

Suitable appetite-regulating agents (appetite suppressants) comprise sibutramine or the mono- and bisdemethylated active metabolites of sibutramine; fenfluramine or dexfenfluramine; mazindol, diethylpropion or phentermine; leptin or modified leptin; dexamphetamine and amphetamine.

Suitable lipase inhibitors comprise orlistat, panclicins, lipase inhibitors isolated from micro organisms such as lipstatin (from Streptomyces toxytricini), ebelactone B (from Streptomyces aburaviensis), synthetic derivatives of these compounds; 2-oxy-4H-3,1-benzoxazin-4-one derivatives like Alizyme's ATL-962 or structurally related compounds; 2-amino-4H-3,1-benzoxazin-4-one derivatives or extracts of plants known to possess lipase inhibitory activity, e.g. extracts of Alpinia officinarum or compounds isolated from such extracts like 3-methylethergalangin (from A. officinarum);

Suitable CB₁-cannabinoid antagonists include rimonabant, SLV319, SR147778 and CP-945598.

Suitable cardiovascular active agents comprise angiotensin II receptor antagonists, e.g. abitesartan, benzyllosartan, candesartan, elisartan, embusartan, enoltasosartan, eprosartan, fonsartan, forasartan, glycyllosartan, irbesartan, isoteoline, losartan, milfasartan, olmesartan, opomisartan, pratosartan, ripisartan, saprisartan, saralasin, sarmesin, tasosartan, telmisartan, valsartan, zolasartan; Kissei KRH-94, Lusofarmaco LR-B/057, Lusofarmaco LR-B/081, Lusofarmaco LR B/087, Searle SC-52458, Sankyo CS-866, Takeda TAK-536, Uriach UR-7247, A-81282, A-81988, BIBR-363, BIBS39, BIBS-222, BMS-180560, BMS-184698, CGP-38560A, CGP-48369, CGP-49870, CGP-63170, CI-996, CV-11194, DA-2079, DE-3489, DMP-811, DuP-167, DuP-532, GA-0056, E-4177, EMD-66397, EMD-73495, EXP-063, EXP-929, EXP-3174, EXP-6155, EXP-6803, EXP-7711, EXP-9270, FK-739, HN-65021, HR-720, ICI-D6888, ICI-D7155, ICI-D8731, KRI-1177, KT3-671, KW-3433, L-158809, L-158978, L-159282, L-159689, L-159874, L-161177, L-162154, L-162234, L-162441, L-163007, L-163017, LY-235656, LY-285434, LY-301875, LY-302289, LY-315995, ME-3221, PD-123177, PD-123319, PD-150304, RG-13647, RWJ-38970, RWJ-46458, S-8307, S-8308, SL-91.0102, U-96849, U-97018, UP-269-6, UP-275-22, WAY-126227, WK-1492.2K, WK-1360, X-6803, XH-148, XR-510, YM-358, YM-31472, ZD-6888, ZD-7155 and ZD-8731 or any physiologically compatible salts, solvates, prodrugs or esters thereof; daglutril; non-selective alpha-adrenoceptor antagonists, e.g. tolazoline or phenoxybenzamine; selective alpha-adrenoceptor antagonists, e.g. doxazosin, prazosin, terazosin or urapidil; beta-adrenoceptor antagonists, e.g. acebutolol, alprenolol, atenolol, betaxolol, bisoprolol, bupranolol, carazolol, carteolol, celiprolol, mepindolol, metipranolol, metoprolol, nadolol, oxprenolol, penbutolol, pindolol, propranolol, sotalol and timolol; mixed antagonists of alpha- and beta-adrenoceptors, e.g. carvedilol or labetolol; ganglion blockers, e.g. reserpine or guanethidine; alpha2-adrenoceptor agonists (including centrally acting alpha2-adrenoceptor agonists), e.g. clonidine, guanfacine, guanabenz methyldopa and moxonidine; renin-inhbitors, e.g. alskiren; ACE-inhbitors, e.g. benazepril, captopril, cilazapril, enalapril, fosinopril, imidapril, lisinopril, moexipril, quinapril, perindopril, ramipril, spirapril or trandolapril; mixed or selective endothelin receptor antagonists e.g. atrasentan, bosentan, clazosentan, darusentan, sitaxsentan, tezosentan, BMS-193884 or J-104132; direct vasodilators, e.g. diazoxide, dihydralazine, hydralazine or minoxidil; mixed ACE/NEP-inhbitors, e.g. omapatrilat; ECE-inhbitors, e.g. FR-901533; PD-069185; CGS-26303; CGS-34043; CGS-35066; CGS-30084; CGS-35066; SM-19712; Ro0677447; selective NEP-inhibitors; vasopressin antagonists, aldosterone receptor antagonists, e.g. eplerenone or spironolactone; angiotensin vaccine; and urotensin II receptor antagonists.

Suitable diuretics comprise thiazide diuretics, e.g. althiazide, bemetizide, bendroflumethiazide, benzylhydrochlorothiazide, benzthiazide, buthiazide, chlorothiazide, cyclothiazide, hydrochlorothiazide, hydroflumethiazide, methyclothiazide, paraflutizide, polythiazide, teclothiazide, trichlormethiazide; thiazide analogue diuretics, e.g. chloraminofenamide, chlortalidone, clofenamide, clopamide, clorexolone, fenquizone, indapamide, mefruside, metolazone, quinethazone, tripamide, xipamide; loop diuretics, e.g. azosemide, bumetanide, furosemide, piretanide, torsemide; potassium sparing diuretics, e.g. amiloride, potassium canrenoate, spironolactone, triamterene or any physiologically compatible tautomers, salts, solvates, prodrugs or esters of any afore mentioned diuretic.

Suitable active agents which alter lipid levels comprise compounds which alter lipid metabolism, such as antihyperlipidemic active ingredients and antilipidemic active ingredients like HMGCoA reductase inhibitors, e.g. atorvastatin, berivastatin, cerivastatin, crilvastatin, fluvastatin, glenvastatin, lovastatin, mevastatin, pitavastatin, pravastatin, rosuvastatin, simvastatin or any physiologically compatible salts, solvates, prodrugs or esters thereof; inhibitors of cholesterol transport/of cholesterol uptake; inhibitors of bile acid reabsorption or inhibitors of the microsomal triglyceride transfer protein (MTP); compounds which reduce food intake, PPAR (=peroxisome proliferator-activated receptors) and RXR agonists and active agents which act on the ATP-dependent potassium channel of the beta cells; fibric acids, e.g. bezafibrate, ciprofibrate, clofibrate, fenofibrate or gemfibrozil; cholestyramine, colestipol, probucol, ezetimibe and dextrothyroxine; HMGCoA synthase inhibitor, a cholesterol absorption inhibitor, an acyl coenzyme A-cholesterol acyl transferase (ACAT) inhibitor, a cholesteryl ester transfer protein (CETP) inhibitor, a squalene synthetase inhibitor, an anti-oxidant, a PPAR α agonist, a FXR receptor modulator, a LXR receptor agonist, a lipoprotein synthesis inhibitor, a renin angiotensin system inhibitor, a microsomal triglyceride transport inhibitor, a bile acid reabsorption inhibitor, a PEAR8 agonist, a triglyceride synthesis inhibitor, a transcription modulator, a squalene epoxidase inhibitor, a low density lipoprotein receptor inducer, a platelet aggregation inhibitor, a 5-LO or FLAP inhibitor, a PPAR 8 partial agonist, and niacin or a niacin receptor agonist, and pharmaceutically acceptable salts and esters thereof.

Further active agents which may be suitable for use in combination with the compound of Formula I according to the present invention may be selected from the group consisting of CART agonists, H3 antagonists, TNF agonists, CRF agonists, CRF BP antagonists, urocortin agonists, beta3-agonists, MSH (melanocyte-stimulating hormone) agonists, serotonin-reuptake inhibitors, mixed serotonin- and noradrenaline-reuptake inhibitors, 5HT modulators, MAO inhibitors, galanin antagonists, growth hormone, growth hormone-releasing compounds, TRH agonists, modulators of uncoupling proteins 2 or 3, leptin agonists, dopamine agonists (bromocriptine, doprexin), RXR modulators, hCNTF agonists and TR-beta-agonists.

Preferred pharmaceutical combination compositions according to the invention comprise combinations of at least one compound of Formula I and at least one biguanide; at least one compound of Formula I and at least one fibric acid; at least one compound of Formula I and at least one HMGCoA reductase inhibitor; and at least one compound of Formula I and at least one insulin sensitizer.

Preferred compounds of Formula I for combination with one or more of the above mentioned active agents are 4-phenyl-piperazine-1-sulfonic acid amide; 4-(2-chloro-phenyl)-piperazine-1-sulfonic acid amide; 4-(2-methoxy-phenyl)-piperazine-1-sulfonic acid amide; 4-pyridin-4-yl-piperazine-1-sulfonic acid amide; 4-pyrimidin-2-yl-piperazine-1-sulfonic acid amide; 4-(4-fluoro-phenyl)-piperazine-1-sulfonic acid amide; 4-(4-chloro-3-trifluoromethyl-phenyl)-piperazine-1-sulfonic acid amide and/or 4-(3-chloro-5-trifluoromethyl-pyridin-2-yl)-piperazine-1-sulfonic acid amide.

Metformine is the preferred biguanide for combination with at least one compound of Formula I.

Preferred fibric acids for combination with at least one compound of Formula I are bezafibrate, ciprofibrate, clofibrate, fenofibrate and/or gemfibrozil. Fenofibrate is most preferred.

Preferred HMGCoA reductase inhibitors for combination with at least one compound of Formula I are atorvastatin, berivastatin, cerivastatin, crilvastatin, fluvastatin, glenvastatin, lovastatin, mevastatin, pitavastatin, pravastatin, rosuvastatin and/or simvastatin or any physiologically compatible salts, solvates, prodrugs or esters thereof. Most preferred are simvastatin, lovastatin and/or pravastatin.

Preferred insulin sensitizers for combination with at least one compound of Formula I are thiazolidinediones, in particular troglitazone, ciglitazone, pioglitazone and/or rosiglitazone. Rosiglitazone and pioglitazone are most preferred.

More preferred combinations according to the invention are the combinations of 4-phenyl-piperazine-1-sulfonic acid amide with metformine; 4-phenyl-piperazine-1-sulfonic acid amide with fenofibrate; 4-phenyl-piperazine-1-sulfonic acid amide with simvastatin and 4-phenyl-piperazine-1-sulfonic acid amide with rosiglitazone.

In one embodiment of the pharmaceutical combination compositions as described above and according to the invention, the compounds of Formula I can be obtained and administered together with the different active agents, e.g. in one combined unit dosage form like in one tablet or capsule, i.e. in a physical combination. In such a combined unit dosage form, the compound of Formula I and the different active agents can be segregated from each other, e.g. by means of different layers in said tablet, e.g. by the use of inert intermediate layers known in the art; or by means of different compartments in said capsule. The corresponding active agents or their pharmaceutically acceptable salts may also be used in form of their hydrates or include other solvents used for crystallization. A unit dosage form may be a fixed combination. A unit dosage form, in particular a fixed combination of the compound of Formula I and one or more of the different active agents is a preferred alternative of this embodiment.

In another embodiment the compounds of Formula I and the different active agents can be obtained and administered in two or more separate unit dosage forms, e.g. in two or more tablets or capsules, the tablets or capsules being physically segregated from each other. The two or more separate unit dosage forms can be administered simultaneously or stepwise (separately), e.g. sequentially one after the other in either order. Thus, the compounds of Formula I and the different active agents can be administered in either order at the same time or at different times spread over the day, the optimal dosage regimen usually being determined by prescription of a physician.

The following examples are intended to explain the invention further, without limiting its scope.

### EXAMPLE 1

### N-Sulfamoyl-N′-phenylpiperazine (=4-Phenyl-piperazine-1-sulfonic acid amide)

A mixture of 25.0 g phenyl-piperazine in 77.0 ml of toluene and 17.8 g sulfamide was allowed to reflux for 8 hours. The mixture was left at room temperature over the weekend. The resulting solids were suspended in 200 ml of methanol and maintained at 90° C. for 60 minutes. The suspension was concentrated (−140 ml methanol) by evaporation under reduced pressure, cooled and filtrated, washed with diethylether and finally dried. The crude product was recovered and recrystallized from 200 ml methanol (procedure above without concentration).

### EXAMPLE 2

### N-Sulfamoyl-N′-phenylpiperazine hydrochloride

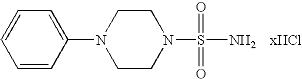

The crystalline fraction as obtained in Example 1 above was treated with ethanolic hydrochloric acid, evaporated and finally dissolved in 100 ml methanol at 65° C. 150 ml of isopropylalcohol were added to this receiving solution and the methanol was removed under reduced pressure. Crystallization overnight, filtration, washing with diethylether and drying under reduced pressure (oil pump) yielded 29.1 g of the title compound, mp.=184° C.

TABLE 8
Elemental analysis of the compound of Example 2 (MW 277.77):

| | calculated | found |
|---|---|---|
| C % | 43.24 | 43.32 |
| H % | 5.81 | 5.86 |
| N % | 15.13 | 15.31 |
| Cl⁻ % | 12.76 | 12.77 |

### EXAMPLE 3

### 4-(2-Methoxy-phenyl)-piperazine-1-sulfonic acid amide hydrochloride

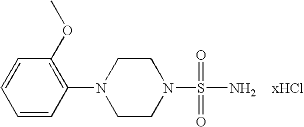

A) 12 ml of chlorosulfonyl isocyanate was added dropwise to an ice-cold solution of 13 ml tert.-butylalcohol in 100 ml dichloromethane. After 30 minutes, 4-dimethylaminopyridine (34.5 g) was added. The resulting mixture was stirred for 1 hour at room temperature and diluted with dichloromethane until a clear solution resulted. This was washed several times with water, the organic layer separated, dried over Na₂SO₄, filtrated and largely evaporated. The residue was recrystallized from acetonitrile to yield 30.4 g of the BOC-protected DMAP-reagent, mp. 156° C.

B) 2-Methoxy-phenyl-piperazine (152 mg) was dissolved in 10 ml of dichloromethane. To this receiving solution, the BOC-protected DMAP-reagent as obtained above (238 mg) was added and the resulting mixture left overnight at room temperature. The mixture was then evaporated and the residue purified by flash-chromatography (stationary phase: silica gel; mobile phase: tetrahydrofurane+5% methanol) to yield 192 mg of the boc-protected intermediate.

C) 100 ml of absolute ethanol were cooled to 0° C. in an ice-bath before 20 ml of acetylchloride were added dropwise and the resulting mixture was stirred for 20 minutes. From the so prepared ethanolic hydrochloric acid solution, 5 ml were separated, the boc-protected compound as obtained above (192 mg) dissolved therein and stirred at room temperature for 3 hours. The mixture was then evaporated several times with ethanol, finally until dryness, to yield 225 mg of the title compound, mp. 191° C.

### EXAMPLE 4

### 4-Pyridin-2-yl-piperazine-1-sulfonic acid amide

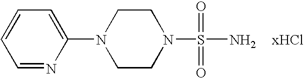

A) Tert.-butyl alcohol (6.5 ml) were dissolved in 30 ml dichloromethane. The resulting solution was added dropwise to an ice-cooled solution of chlorosulfonyl isocyanate (6.0 ml) in 40 ml dichloromethane. After 30 min reaction time, the resulting mixture was diluted with dichloromethane up to 100 ml to get a 0.854 molar stock solution which was used for the next step without further purification.

B) A freshly prepared stock solution of tert.-butylsulfamoylchloride (1.67 ml; 0.854 molar in dichloromethane, for preparation see above) was added to a solution of 2-pyridinyl-piperazine (232 mg) in 4 ml dichloromethane and the resulting mixture was stirred at room temperature for 24 hours. 3 ml of an ethanolic HCl solution (for preparation see ex. 3C, above) were then added, before the resulting mixture was left over night at room temperature. After evaporation of the solvents under reduced pressure, a crude solid was isolated. Subsequent flash chromatography of this solid (stationary phase: silicagel; mobile phase: tetrahydrofuran/methanol/ammonia 70:30:1 v/v/v) and drying of the product fractions yielded 200 mg of the title compound; liquid chromatography mass spectroscopy (=LC-MS): M+H 243 (99% ELSD).

### EXAMPLE 5

### 4-(4′-Fluoro-biphenyl-4-yl)-piperazine-1-sulfonic acid amide

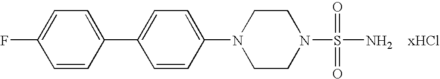

A) 1-(4-Bromophenyl)-piperazine (250 mg), 4-fluorobenzeneboronic acid (254 mg), potassium carbonate (372 mg, dried and grinded) and palladium-(II)-acetate (23.3 mg) were dissolved in 20 ml of a mixture of ethylene glycol dimethyl ether/water/ethanol (7:3:2 v/v/v) and put into a microwave reactor (Emrys Optimizer®). After 5 min. reaction time at 150° C., methyl tert.-butyl ether was added to the now clear solution, the organic phase was washed consecutively with water and brine and dried over Na₂SO₄. The organic phase was largely evaporated under reduced pressure. Another equal batch was produced and the crude products of both batches were together dissolved in dichloromethane. The organic phase was washed with diluted soda solution, dried over Na₂SO₄, the solvent largely evaporated under reduced pressure and the residue purified by flash chromatography (stationary phase: silica gel, mobile phase: dichloromethane/methanol 9:1 v/v) to yield 0.6 g of 1-(4′-fluoro-biphenyl-4-yl)-piperazine.

¹H-NMR (500 MHz), δ [ppm]: 7.62 d (1H), 7.61 d (1H), 7.22 t (2 H), 7.49 d (2H), 6.98 d (2H), 3.17 m (4H), 3.09 m (4H).

B) 1-(4′-Fluoro-biphenyl-4-yl)-piperazine (0.6 g) as obtained above, and sulfamide (0.3 g) were dissolved in 30 ml of dioxane and then heated under reflux cooling for 3 hours. After cooling to room temperature, the solvent was largely removed under reduced pressure. The resulting solid was crystallized from methyl tert.-butyl ether to yield 0.4 g of the title compound, m.p. 243.5-245.2° C.

The compounds of Formula I listed in the following Table 9 can also be prepared according to the processes described in the foregoing examples or according to processes analogous thereto:

TABLE 9
Further compounds of Formula I

| Example | | | |
|---|---|---|---|
| No. | Ar | Salt | m.p. |
| 6 | 4-pyridinyl | HCl | 243° C. |
| 7 | 2-pyrimidinyl | | |
| 8 | 2,3-dimethylphenyl | HCl | 202° C. |
| 9 | 4-fluorophenyl | HCl | 125° C. |
| 10 | 3-chlorophenyl | | |
| 11 | 2-methyl-5-chlorophenyl | HCl | 190° C. |
| 12 | 3-trifluoromethyl-4-chlorophenyl | HCl | 180° C. |
| 13 | 3-cyano-2-pyridinyl | HCl | 190° C. |
| 14 | 3-chloro-5-trifluoro-2-pyridinyl | HCl | 159° C. |
| 15 | 4-acetylphenyl | HCl | 156° C. |
| 16 | 3,5-dichloro-4-pyridinyl | HCl | 206° C. |
| 17 | 2-trifluoromethyl-4-quinolyl | HCl | 191° C. |
| 18 | 4-trifluoro-2-pyrimidinyl | HCl | 156° C. |
| 19 | 5-trifluoro-2-pyridinyl | HCl | 169° C. |
| 20 | 2-nitro-4-trifluorophenyl | HCl | 134° C. |
| 21 | 2-fluoro-4-methylsulfonylphenyl | HCl | 184° C. |
| 22 | benzo[1,3]dioxol-5-ylmethyl | HCl | 233° C. |
| 23 | 1-naphthalenyl | HCl | 193° C. |
| 24 | 4-ethoxyphenyl | | |
| 25 | 5,6-dimethyl-thieno[2,3-D]-4-pyrimidinyl | | |
| 26 | 2-methyl-mercaptophenyl | | |
| 27 | 2-(tert.-butyl)-5-(trifluoromethyl)pyrazolo- | | |
| | [1,5-A]-7-pyrimidinyl | | |
| 28 | benzyloxyphenyl | | |
| 29 | 5-cyano-6-methyl-2-nicotinic acid ethyl ester | | |
| 30 | 3,5-dichlorophenyl | HCl | 185° C. |
| 31 | 3,4-dichlorophenyl | HCl | 176° C. |
| 32 | 2,4-difluorophenyl | HCl | 165° C. |
| 33 | 4-trifluorophenyl | HCl | 159° C. |

### EXAMPLE I

### Capsules containing N-sulfamoyl-N′-phenylpiperazine hydrochloride

Capsules with the following composition per capsule were produced:

| | |
|---|---|
| N-Sulfamoyl-N′-phenylpiperazine hydrochloride | 70 mg |
| Corn starch | 60 mg |
| Lactose | 250 mg |
| Ethylacetate (= EA) | q.s. |

The active substance, the corn starch and the lactose are processed into a homogeneous pasty mixture using EA. The paste is ground and the resulting granules are placed on a suitable tray and dried at 45° C. in order to remove the solvent. The dried granules are passed through a crusher and mixed in a mixer with the further following auxiliaries:

| | |
|---|---|
| Talcum | 5 mg |
| Magnesium stearate | 5 mg |
| Corn starch | 10 mg |

and are then poured into 400 mg capsules (=capsule size 0).

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of treating or inhibiting obesity in a human or other mammal subject, said method comprising administering to said subject an effective amount of a compound corresponding to Formula I 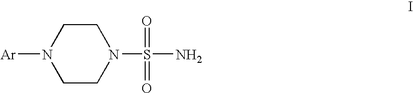
wherein
Ar is monocyclic or bicyclic C₆₋₁₀-aryl,
whose ring carbon atoms are optionally replaced one to three times by nitrogen, oxygen and/or sulfur, and/or
whose C₆₋₁₀-aryl ring system optionally contains three to five double bonds, and/or
whose C₆₋₁₀-aryl ring system is optionally substituted by one, two or three substituents which may be the same or different and which may be selected from the group consisting of halogen, carboxy, hydroxy, hydroxycarbamoyl, trifluoromethyl, cyano, nitro, pyrrolidinyl, C₁₋₄-alkyl, C₁₋₄-alkoxy, C₀₋₄-alkoxyphenyl, C₁₋₄-alkylthio, C₂₋₄-alkanoyl, C₁₋₄-alkyloxycarbonyl, C₁₋₄-alkylsulfonyl; and two oxygen atoms which are bonded to two adjacent carbon atoms of the C₆₋₁₀-aryl ring system and which are bridged by C₁₋₂-alkylene; or
whose C₆₋₁₀-aryl ring system is substituted by one or two substituents which may be the same or different and which may be selected from the group consisting of halogen, carboxy, hydroxy, hydroxycarbamoyl, trifluoromethyl, cyano, nitro, C₁₋₄-alkyl, C₁₋₄-alkoxy, C₁₋₄-alkylthio, C₂₋₄-alkanoyl, C₁₋₄-alkyloxycarbonyl, C₁₋₄-alkylsulfonyl; two oxygen atoms which are bonded to two adjacent carbon atoms of the C₆₋₁₀-aryl ring system and which are bridged by C₁₋₂-alkylene; or
whose C₆₋₁₀-aryl ring system is substituted by thienyl, naphthyl, pyridinyl; phenyl or benzyl, each of which phenyl or benzyl being optionally substituted in the phenyl ring by one, two or three substituents which may be the same or different and which may be selected from halogen, trifluoromethyl, cyano, C₁₋₆-alkyl, C₁₋₄-alkoxy or C₁₋₄-alkylsulfonyl;
or a physiologically compatible salt thereof.

2. A method according to claim 1, wherein
Ar is phenyl, optionally substituted by one, two or three substituents which may be the same or different and which may be selected from the group consisting of halogen, carboxy, hydroxy, hydroxycarbamoyl, trifluoromethyl, cyano, nitro, C₁₋₄-alkyl, C₁₋₄-alkoxy, C₀₋₄-alkoxyphenyl, C₁₋₄-alkylthio, C₂₋₄-alkanoyl, C₁₋₄-alkyloxycarbonyl, C₁₋₄-alkylsulfonyl and two oxygen atoms bonded to adjacent carbon atoms which are bridged by C₁₋₂-alkylene; or is phenyl substituted by phenyl or benzyl, each of which optionally being substituted in the phenyl ring by one or two substituents which may be the same or different and which may be selected from halogen, trifluoromethyl, C₁₋₄-alkyl and C₁₋₄-alkoxy; or
is naphthyl; pyridinyl; pyrimidinyl; pyrazinyl; pyridazinyl; triazinyl; quinolinyl; isoquinolinyl; 1,2,3,4-tetrahydroisoquinolinyl; indolyl; isoindolinyl; thieno[3,2-d]pyrimidinyl or pyrazolo[1,5-a]pyrimidinyl, each being optionally substituted by one or two substituents which may be the same or different and which may be selected from the group consisting of halogen, hydroxycarbamoyl, trifluoromethyl, cyano, nitro, pyrrolidinyl, C₁₋₄-alkyl, C₁₋₄-alkoxy and C₁₋₄-alkyloxycarbonyl.

3. A method according to claim 1, wherein
Ar is phenyl, optionally substituted by one or two substituents which may be the same or different and which may be selected from the group consisting of halogen, hydroxy, hydroxycarbamoyl, trifluoromethyl, cyano, nitro, C₁₋₄-alkyl, C₁₋₄-alkoxy, C₂₋₄-alkanoyl, C₁₋₄-alkyloxycarbonyl, C₁₋₄-alkylsulfonyl and two oxygen atoms bonded to adjacent carbon atoms which are bridged by C₁₋₂-alkylene; or
is pyridinyl; pyrimidinyl; naphthyl; quinolinyl; isoquinolinyl; 1,2,3,4-tetrahydroisoquinolinyl; indolyl or isoindolinyl, each optionally being substituted by one or two substituents which may be the same or different and which may be selected from the group consisting of halogen, hydroxycarbamoyl, trifluoromethyl, cyano, nitro, C₁₋₄-alkyl, C₁₋₄-alkoxy and C₁₋₄-oxycarbonyl.

4. A method according to claim 1, wherein
Ar is phenyl substituted by one or two substituents which may be the same or different and which may be selected from the group consisting of halogen, hydroxy, trifluoromethyl, cyano, nitro, C₁₋₄-alkyl, C₁₋₄-alkoxy, C₂₋₄-alkanoyl, C₁₋₄-alkylsulfonyl and two oxygen atoms bonded to adjacent carbon atoms which are bridged by C₁₋₂-alkylene; or
is pyridinyl; pyrimidinyl or quinolinyl; each optionally being substituted by one or two substituents which may be the same or different and which may be selected from the group consisting of halogen, trifluoromethyl, cyano, nitro, C₁₋₄-alkyl and C₁₋₄-alkoxy.

5. A method of treating at least one condition selected from the group consisting of metabolic syndrome, syndrome X, cardiovascular disease, diabetic conditions or diseases unrelated to obesity, and epilepsy in a human or mammal subject in need thereof, said method comprising administering to said subject an effective amount of a compound according to claim 1.

6. A method according to claim 5, wherein said at least one condition comprises at least one metabolic syndrome or syndrome X disorder or disease selected from the group consisting of hypertension, insulin resistance, glucose intolerance, dyslipoproteinaemia, and hyperuricaemia.

7. A method according to claim 6, wherein said at least one metabolic syndrome or syndrome X disorder or disease comprises arterial hypertension, diabetes mellitus type II, or hypertriglyceridaemia accompanied by dyslipoproteinaemia occurring with lowered HDL-cholesterol.

8. A method according to claim 5, wherein said condition is a cardiovascular disease selected from the group consisting of coronary heart disease, cerebrovascular diseases and peripheral occlusive arterial disease.

9. A method according to claim 5, wherein said condition is diabetic conditions or diseases unrelated to obesity.

10. A method according to claim 5, wherein said condition is epilepsy.

11. A compound corresponding to formula Ia 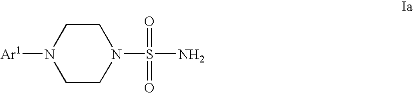
wherein
Ar¹ is phenyl, optionally substituted by one, two or three substituents which may be the same or different and which may be selected from the group consisting of halogen, carboxy, hydroxy, hydroxycarbamoyl, trifluoromethyl, cyano, nitro, C₁₋₄-alkyl, C₁₋₄-alkoxy, C₀₋₄-alkoxyphenyl, C₁₋₄-alkylthio, C₂₋₄-alkanoyl, C₁₋₄-alkyloxycarbonyl, C₁₋₄-alkylsulfonyl and two oxygen atoms bonded to adjacent carbon atoms which are bridged by C₁₋₂-alkylene; or is phenyl substituted by phenyl or benzyl, each of which optionally being substituted in the phenyl ring by one or two substituents which may be the same or different and which may be selected from halogen, C₁₋₄-alkyl, C₁₋₄-alkoxy and trifluoromethyl; or
is naphthyl; pyridinyl; 2-pyrimidinyl; 5-pyrimidinyl; pyrazinyl; pyridazinyl; triazinyl; quinolinyl; isoquinolinyl; 1,2,3,4-tetrahydroisoquinolinyl; indolyl; isoindolinyl; thieno[3,2-d]pyrimidinyl or pyrazolo[1,5-a]pyrimidinyl each optionally being substituted by one or two substituents which may be the same or different and which may be selected from the group consisting of halogen, hydroxycarbamoyl, trifluoromethyl, cyano, nitro, pyrrolidinyl, C₁₋₄-alkyl, C₁₋₄-alkoxy and C₁₋₄-alkyloxycarbonyl;
or a physiologically compatible salt thereof.

12. A compound according to claim 11, wherein
Ar¹ is phenyl, optionally substituted by one or two substituents which may be the same or different and which may be selected from the group consisting of halogen, hydroxy, hydroxycarbamoyl, trifluoromethyl, cyano, nitro, C₁₋₄-alkyl, C₁₋₄-alkoxy, C₂₋₄-alkanoyl, C₁₋₄-alkyloxycarbonyl, C₁₋₄-alkylsulfonyland two oxygen atoms bonded to adjacent carbon atoms which are bridged by C₁₋₂-alkylene; or
is naphthyl; pyridinyl; 2-pyrimidinyl; 5-pyrimidinyl; quinolinyl; isoquinolinyl; 1,2,3,4-tetrahydroisoquinolinyl; indolyl or isoindolinyl, each optionally being substituted by one or two substituents which may be the same or different and which may be selected from the group consisting of halogen, hydroxycarbamoyl, trifluoromethyl, cyano, nitro, C₁₋₄-alkyl, C₁₋₄-alkoxy and C₁₋₄-alkyloxycarbonyl.

13. A compound according to claim 11, wherein
Ar¹ is phenyl, optionally substituted by one or two substituents which may be the same or different and which may be selected from the group consisting of halogen, hydroxy, trifluoromethyl, cyano, nitro, C₁₋₄-alkyl, C₁₋₄-alkoxy, C₂₋₄-alkanoyl, C₁₋₄-alkylsulfonyl and two oxygen atoms bonded to adjacent carbon atoms which are bridged by C₁₋₂-alkylene; or
is pyridinyl; 2-pyrimidinyl; 5-pyrimidinyl or quinolinyl; each optionally being substituted by one or two substituents which may be the same or different and which may be selected from the group consisting of halogen, trifluoromethyl, cyano, nitro, C₁₋₄-alkyl and C₁₋₄-alkoxy.

14. A compound according to claim 11, wherein
Ar¹ is phenyl substituted by one or two substituents which may be the same or different and which may be selected from the group consisting of halogen, trifluoromethyl, C₁₋₄-alkyl, C₁₋₄-alkoxy and C₁₋₄-alkylsulfonyl; or
is pyridinyl; 2-pyrimidinyl; 5-pyrimidinyl or quinolinyl; each optionally being substituted by one or two substituents which may be the same or different and which may be selected from the group consisting of halogen, trifluoromethyl, cyano C₁₋₄-alkyl and C₁₋₄-alkoxy.

15. A compound according to claim 11, selected from the group consisting of:
4-phenyl-piperazine-1-sulfonic acid amide;
4-(2-chloro-phenyl)-piperazine-1-sulfonic acid amide; and
4-(2-methoxy-phenyl)-piperazine-1-sulfonic acid amide.

16. A pharmaceutical composition comprising a compound according to claim 11 and a pharmaceutically acceptable auxiliary or carrier.

17. A compound corresponding to Formula Ib 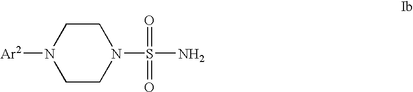
wherein
Ar² is phenyl substituted once by fluoro, 3-chloro, 4-chloro, bromo, iodo, hydroxy, C₁₋₄-alkyl, C₂₋₄-alkoxy, C₀₋₄-alkoxyphenyl, C₁₋₄-alkylthio, C₂₋₄-alkanoyl, C₁₋₄-oxycarbonyl, hydroxycarbamoyl, carboxy, trifluoromethyl, cyano, nitro, two oxygen atoms bonded to adjacent carbon atoms which are bridged by C₁₋₂-alkylene, and C₁₋₄-alkylsulfonyl; or
is phenyl substituted by two or three substituents which may be the same or different and which may be selected from the group consisting of halogen, carboxy, hydroxy, hydroxycarbamoyl, trifluoromethyl, cyano, nitro, C₁₋₄-alkyl, C₁₋₄-alkoxy, C₁₋₄-alkylthio, C₂₋₄-alkanoyl, C₁₋₄-oxycarbonyl, C₁₋₄-alkylsulfonyl and two oxygen atoms bonded to adjacent carbon atoms which are bridged by C₁₋₂-alkylene; or
is phenyl substituted once by phenyl or benzyl, each of which optionally being substituted in the phenyl ring by one or two substituents which may be the same or different and which may be selected from halogen, trifluoromethyl, C₁₋₄-alkyl and C₁₋₄-alkoxy; or
is naphthyl; pyridinyl; 2-pyrimidinyl; 5-pyrimidinyl; pyrazinyl; pyridazinyl; triazinyl; quinolinyl; isoquinolinyl; indolyl; isoindolinyl; thieno[3,2-d]pyrimidinyl or pyrazolo[1,5-a]pyrimidinyl each optionally being substituted by one or two substituents which may be the same or different and which may be selected from the group consisting of halogen, hydroxycarbamoyl, trifluoromethyl, cyano, nitro, pyrrolidinyl, C₁₋₄-alkyl, C₁₋₄-alkoxy and C₁₋₄-oxycarbonyl; or
is 1,2,3,4-tetrahydroisoquinolinyl substituted by one or two substituents which may be the same or different and which may be selected from the group consisting of halogen, hydroxycarbamoyl, trifluoromethyl, cyano, nitro, pyrrolidinyl, C₁₋₄-alkyl, C₁₋₄-alkoxy and C₁₋₄-oxycarbonyl;
or a physiologically compatible salt thereof.

18. A compound according to claim 17, wherein
Ar² is phenyl substituted once by fluoro, 3-chloro, 4-chloro, bromo, iodo, hydroxy, trifluoromethyl, cyano, nitro, C₁₋₄-alkyl, C₂₋₄-alkoxy, C₂₋₄-alkanoyl, C₁₋₄-alkylsulfonyl and two oxygen atoms bonded to adjacent carbon atoms which are bridged by C₁₋₂-alkylene; or
is phenyl substituted by two substituents which may be the same or different and which may be selected from the group consisting of halo, hydroxy, trifluoromethyl, cyano, nitro, C₁₋₄-alkyl, C₁₋₄-alkoxy, C₂₋₄-alkanoyl, C₁₋₄-alkylsulfonyl and two oxygen atoms bonded to adjacent carbon atoms which are bridged by C₁₋₂-alkylene; or
is pyridinyl; 2-pyrimidinyl; 5-pyrimidinyl; naphthyl; quinolinyl; isoquinolinyl; indolyl or isoindolinyl, each optionally being substituted by one or two substituents which may be the same or different and which may be selected from the group consisting of halogen, trifluoromethyl, cyano, nitro, C₁₋₄-alkyl and C₁₋₄-alkoxy; or
is 1,2,3,4-tetrahydroisoquinolinyl substituted by one or two substituents which may be the same or different and which may be selected from the group consisting of halogen, trifluoromethyl, cyano, nitro, C₁₋₄-alkyl and C₁₋₄-alkoxy.

19. A compound according to claim 17, wherein
Ar² is phenyl substituted once by fluoro, 3-chloro, 4-chloro, bromo, iodo, hydroxy, trifluoromethyl, cyano, nitro, C₁₋₄-alkyl, C₂₋₄-alkoxy, C₂₋₄-alkanoyl, C₁₋₄-alkylsulfonyl and two oxygen atoms bonded to adjacent carbon atoms which are bridged by C₁₋₂-alkylene; or
is phenyl substituted by two substituents which may be the same or different and which may be selected from the group consisting of halogen, hydroxy, trifluoromethyl, cyano, nitro, C₁₋₄-alkyl, C₁₋₄-alkoxy, C₂₋₄-alkanoyl, C₁₋₄-alkylsulfonyl and two oxygen atoms bonded to adjacent carbon atoms which are bridged by C₁₋₂-alkylene; or
is pyridinyl; 2-pyrimidinyl; 5-pyrimidinyl; quinolinyl; each optionally being substituted by one or two substituents which may be the same or different and which may be selected from the group consisting of halogen, trifluoromethyl, cyano, nitro, C₁₋₄-alkyl and C₁₋₄-alkoxy.

20. A compound according to claim 17, wherein
Ar² is phenyl substituted once by fluoro, 3-chloro, 4-chloro, bromo, iodo, trifluoromethyl, C₁₋₄-alkyl, C₂₋₄-alkoxy and C₁₋₄-alkylsulfonyl; or
is phenyl substituted by two substituents which may be the same or different and which may be selected from the group consisting of halogen, trifluoromethyl, C₁₋₄-alkyl, C₁₋₄-alkoxy and C₁₋₄-alkylsulfonyl; or
is pyridinyl; 2-pyrimidinyl; 5-pyrimidinyl or quinolinyl; each optionally being substituted by one or two substituents which may be the same or different and which may be selected from the group consisting of halogen, trifluoromethyl, cyano, C₁₋₄-alkyl and C₁₋₄-alkoxy.

21. A compound according to claim 17, selected from the group consisting of:
4-pyridin-4-yl-piperazine-1-sulfonic acid amide;
4-pyrimidin-2-yl-piperazine-1-sulfonic acid amide;
4-(4-fluoro-phenyl)-piperazine-1-sulfonic acid amide;
4-(4-chloro-3-trifluoromethyl-phenyl)-piperazine-1-sulfonic acid amide, and
4-(3-chloro-5-trifluoromethyl-pyridin-2-yl)-piperazine-1-sulfonic acid amide.

22. A method of producing a compound corresponding to Formula I 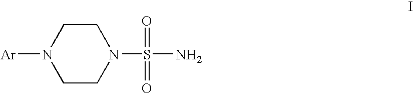
wherein
Ar is monocyclic or bicyclic C₆₋₁₀-aryl,
whose ring carbon atoms are optionally replaced one to three times by nitrogen, oxygen and/or sulfur, and/or
whose C₆₋₁₀-aryl ring system optionally contains three to five double bonds, and/or
whose C₆₋₁₀-aryl ring system is optionally substituted by one, two or three substituents which may be the same or different and which are selected from the group consisting of halogen, carboxy, hydroxy, hydroxycarbamoyl, trifluoromethyl, cyano, nitro, pyrrolidinyl, C₁₋₄-alkyl, C₁₋₄-alkoxy, C₀₋₄-alkoxyphenyl, C₁₋₄-alkylthio, C₂₋₄-alkanoyl, C₁₋₄-alkyloxycarbonyl, C₁₋₄-alkylsulfonyl; and two oxygen atoms which are bonded to two adjacent carbon atoms of the C₆₋₁₀-aryl ring system and which are bridged by C₁₋₂-alkylene; or
whose C₆₋₁₀-aryl ring system is substituted by one or two substituents which may be the same or different and which may be selected from the group consisting of
halogen, carboxy, hydroxy, hydroxycarbamoyl, trifluoromethyl, cyano, nitro, C₁₋₄-alkyl, C₁₋₄-alkoxy, C₁₋₄-alkylthio, C₂₋₄-alkanoyl, C₁₋₄-alkyloxycarbonyl, C₁₋₄-alkylsulfonyl; two oxygen atoms which are bonded to two adjacent carbon atoms of the C₆₋₁₀-aryl ring system and which are bridged by C₁₋₂-alkylene; or
whose C₆₋₁₀-aryl ring system is substituted by thienyl, naphthyl, pyridinyl; phenyl or benzyl, each of which phenyl or benzyl being optionally substituted in the phenyl ring by one, two or three substituents which may be the same or different and which may be selected from halogen, trifluoromethyl, cyano, C₁₋₆-alkyl, C₁₋₄-alkoxy or C₁₋₄-alkylsulfonyl;
and of its physiologically compatible acid addition salts, by either
a) reacting an arylpiperazine compound of general Formula II, 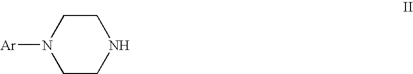
wherein Ar has the above meaning, with sulfamide, or
b) reacting an arylpiperazine of Formula II with a 4-dimethylaminopyridin (=DMAP) reagent which is protected with the tert.-butyloxycarbonyl (=boc) group, of Formula III, 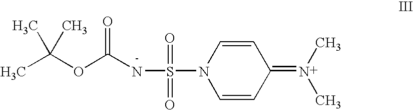
and subsequently cleaving off the boc group under acidic conditions from the obtained intermediate compound, or
c) reacting an arylpiperazine of Formula II with sulfamoylchloride, which is preferably protected with the boc group, of Formula IV, 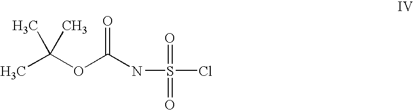
and subsequently cleaving off the boc group under acidic conditions from the obtained intermediate product,
and if desired converting resulting free bases of Formula I into their physiologically compatible salts, or converting salts of the compounds of Formula I into the free bases of Formula I.

23. A pharmaceutical composition comprising:
a) at least one compound of Formula I according to claim 1 as a first active agent, and
b) at least one substance selected from the group consisting of biguanides; fibric acids; HMGCoA reductase inhibitors; and insulin sensitizers as a second active agent.

24. A pharmaceutical composition according to claim 23, further comprising at least one pharmaceutically acceptable auxiliary or carrier.

25. A pharmaceutical composition according to claim 23, wherein the composition is orally administrable and the active agents are present at least one dosage form selected from the group consisting of tablets, coated tablets, capsules, syrups, elixirs, and suspensions.

26. A pharmaceutical composition according to claim 21, wherein said compound of Formula I according to claim 1 is selected from the group consisting of:
4-phenyl-piperazine-1-sulfonic acid amide;
4-(2-chloro-phenyl)-piperazine-1-sulfonic acid amide;
4-(2-methoxy-phenyl)-piperazine-1-sulfonic acid amide;
4-pyridin-4-yl-piperazine-1-sulfonic acid amide;
4-pyrimidin-2-yl-piperazine-1-sulfonic acid amide;
4-(4-fluoro-phenyl)-piperazine-1-sulfonic acid amide;
4-(4-chloro-3-trifluoromethyl-phenyl)-piperazine-1-sulfonic acid amide, and
4-(3-chloro-5-trifluoromethyl-pyridin-2-yl)-piperazine-1-sulfonic acid amide.

27. A pharmaceutical composition according to claim 26, wherein the compound of Formula I is 4-phenyl-piperazine-1-sulfonic acid amide.

28. A pharmaceutical composition according to claim 23, wherein the second active agent is a biguanide or a physiologically compatible salt, solvate, prodrug or ester thereof.

29. A pharmaceutical composition according to claim 28, wherein the second active agent is metformine.

30. A pharmaceutical composition according to claim 23, wherein the second active agent is a fibric acid or a physiologically compatible salt, solvate, prodrug or ester thereof.

31. A pharmaceutical composition according to claim 30, wherein the second active agent is fenofibrate.

32. A pharmaceutical composition according to claim 23, wherein the second active agent is a HMGCoA reductase inhibitor or a physiologically compatible salt, solvate, prodrug or ester thereof.

33. A pharmaceutical composition according to claim 32, wherein the second active agent is simvastatin.

34. A pharmaceutical composition according to claim 23, wherein the second active agent is an insulin sensitizer or a physiologically compatible salt, solvate, prodrug or ester thereof.

35. A pharmaceutical composition according to claim 34, wherein the second active agent is rosiglitazone.

36. A method of treating or inhibiting at least one condition selected from the group consisting of obesity, metabolic syndrome, syndrome X, cardiovascular diseases, diabetic conditions or diseases unrelated to obesity, and epilepsy in a human or other mammal subject in need thereof, said method comprising administering to said subject in combination an effective amount of:
a) at least one compound of Formula I according to claim 1 as a first active agent, and
b) at least one substance selected from the group consisting of biguanides; fibric acids; HMGCoA reductase inhibitors, and insulin sensitizers as a second active agent.

37. A kit comprising in separate containers in a single package pharmaceutical dosage forms for use in combination, comprising:
i) in one separate container a pharmaceutical dosage form comprising at least one compound of Formula I, and
ii) in another separate container a pharmaceutical dosage form comprising at least one active agent selected from the group consisting of biguanides; fibric acids; HMGCoA reductase inhibitos; and insulin sensitizers.
